(19) United States
(12) Patent Application Publication
(10) Pub. No.: US 2023/0048598 A1
(43) Pub. Date: Feb. 16, 2023

(54) SYSTEM AND METHOD FOR PROCESSING VIRUS PREPARATIONS TO REDUCE HETEROGENEITY

(71) Applicant: THE TRUSTEES OF INDIANA UNIVERSITY, Bloomington, IN (US)

(72) Inventors: Martin F. JARROLD, Bloomington, IN (US); David E. CLEMMER, Bloomington, IN (US); Benjamin E. DRAPER, Bloomington, IN (US); Lauren F. BARNES, Bloomington, IN (US)

(21) Appl. No.: 17/791,981

(22) PCT Filed: Feb. 3, 2021
(86) PCT No.: PCT/US2021/016325

§ 371 Date: Jul. 11, 2022

Related U.S. Application Data
(60) Provisional application No. 62/969,323, filed on Feb. 3, 2020.

(51) Int. Cl. C12N 7/00; G01N 33/68

(52) U.S. Cl. CPC C12N 7/00; G01N 33/6848; C12N 2750/14152; C12N 2750/14121

(57) ABSTRACT

A method for reducing heterogeneity of a virus preparation may include generating virus ions from the virus preparation, repeatedly increasing at least one of a temperature and an incubation period at the increased temperature of at least one of the virus preparation and the generated virus ions, measuring mass-to-charge ratios and charge magnitudes of at least some of the generated virus ions at each increase of the at least one of the temperature and the incubation period, determining a mass spectrum at each increase of the at least one of the temperature and the incubation period based on values of the respective mass-to-charge ratios and charge magnitudes, and determining, based on the mass spectrums, optimum ones of the temperature and the incubation period which together minimize, or at least reduce, a heterogeneity of the virus preparation without aggregation of virus capsids in the virus preparation.

### CROSS-REFERENCE TO RELATED APPLICATION

This international patent application claims the benefit of, and priority to, U.S. Provisional Patent Application Ser. No. 62/969,323, filed Feb. 3, 2020, the disclosure of which is expressly incorporated herein by reference in its entirety.

### GOVERNMENT RIGHTS

This invention was made with government support under GM131100 awarded by the National Institutes of Health. The United States Government has certain rights in the invention.

### TECHNICAL FIELD

The present disclosure relates generally to mass spectrometry, and more specifically to instruments and methods for measuring and analyzing masses of biological mixture particles including, but not limited to, virus particles, over a range of different temperature, incubation period, heating profile and/or cooling profile.

### BACKGROUND

Adeno-associated virus (AAV) is one example of a gene therapy vector which has gained wide acceptance due to its lack of pathogenicity, low immunogenicity and the existence of many serotypes with different tropisms. There has been found to exist a potential for dose-related immunotoxicity which may be related to sample preparation and packaging techniques. It may be beneficial to treat virus preparations, such as, but not limited to, AAV in a manner which reduces the heterogeneity of such preparations.

### SUMMARY

The present disclosure may comprise one or more of the features recited in the attached claims, and/or one or more of the following features and combinations thereof. In a first aspect, A method for reducing heterogeneity of a virus preparation may comprise generating virus ions from the virus preparation, repeatedly increasing at least one of a temperature and an incubation period at the increased temperature of at least one of the virus preparation and the generated virus ions, measuring mass-to-charge ratios and charge magnitudes of at least some of the generated virus ions at each increase of the at least one of the temperature and the incubation period, determining a mass spectrum at each increase of the at least one of the temperature and the incubation period based on values of the respective mass-to-charge ratios and charge magnitudes, and determining, based on the mass spectrums, optimum ones of the temperature and the incubation period which together minimize, or at least reduce, a heterogeneity of the virus preparation without aggregation of virus capsids in the virus preparation.

A second aspect may include the features of the first aspect, and may further comprise varying a cooling profile corresponding to a manner in which the increased temperature is reduced following the respective incubation period, and determining, based on the mass spectrums, an optimum cooling profile along with the optimum ones of the temperature and the incubation period which, in combination, minimize, or at least reduce, the heterogeneity of the virus preparation without aggregation of virus capsids in the virus preparation.

A third aspect may include the features of the first aspect, and may further comprise varying a heating profile corresponding to a manner in which the temperature of the at least one of the virus preparation and the generated virus ions is increased, and determining, based on the mass spectrums, an optimum heating profile along with the optimum ones of the temperature and the incubation period which, in combination, minimize, or at least reduce, the heterogeneity of the virus preparation without aggregation of virus capsids in the virus preparation.

A fourth aspect may include the features of the third aspect, and may further comprise varying a cooling profile corresponding to a manner in which the increased temperature is reduced following the respective incubation period, and determining, based on the mass spectrums, an optimum cooling profile along with the optimum heating profile and the optimum ones of the temperature and the incubation period which, in combination, minimize, or at least reduce, the heterogeneity of the virus preparation without aggregation of virus capsids in the virus preparation.

A fifth aspect may include the features of any of the first through fourth aspects, wherein measuring mass-to-charge ratios and charge magnitudes of at least some of the generated virus ions at each increase of the at least one of the temperature and the incubation period is carried out using a charge detection mass spectrometer.

A sixth aspect may include the features of any the first through fourth aspects, wherein measuring mass-to-charge ratios and charge magnitudes of at least some of the generated virus ions at each increase of the at least one of the temperature and the incubation period is carried out using a mass spectrometer.

A seventh aspect may include the features of any of the first through sixth aspects, and may further comprise determining the heterogeneity of the virus population at each increase of the at least one of the temperature and the incubation period based on mass resolution of at least one mass peak of interest in the respective mass spectrum.

An eighth aspect may include the features of any of the first through seventh aspects, and may further comprise determining at each increase of the at least one of the temperature and the incubation period that aggregation has occurred if the respective mass spectrum includes discernable particles with masses greater than that of a highest mass capsid in the virus preparation, wherein at least one of the optimum ones of the temperature and the incubation period is less than the respective temperature and incubation period of a respective mass spectrum in which aggregation has occurred.

A ninth aspect may include the features of any of the first through eighth aspects, and may further comprise treating other samples of the virus preparation to minimize, or at least reduce, heterogeneity thereof by heating each of the other samples of the virus preparation to the determined optimum temperature for the optimum incubation period.

A tenth aspect may include the features of any of the first through ninth aspects, wherein the virus preparation is a virus preparation solution, and wherein generating the virus ions comprises generating the virus ions from the virus preparation solution using an electrospray ionization source.

An eleventh aspect may include the features of any of the first through tenth aspects, wherein repeatedly increasing the at least one of the temperature and the incubation period comprises controlling a first thermal energy device coupled to the virus preparation to heat the virus preparation.

A twelfth aspect may include the features of any of the first through eleventh aspects, wherein repeatedly increasing the at least one of the temperature and the incubation period comprises controlling a second thermal energy device, positioned to transfer thermal energy to the generated ions, to heat the generated ions.

In a thirteenth aspect, a method for reducing heterogeneity of a virus preparation may comprise sequentially increasing at least one of a temperature and an incubation period at the increased temperature of the virus preparation, generating virus ions from the virus preparation at each increase of the at least one of the temperature and the incubation period, measuring mass-to-charge ratios and charge magnitudes of at least some of the generated virus ions at each increase of the at least one of the temperature and the incubation period, determining a mass spectrum at each increase of the at least one of the temperature and the incubation period based on values of the respective mass-to-charge ratios and charge magnitudes, and determining, based on the mass spectrums, optimum ones of the temperature and the incubation period which together minimize, or at least reduce, a heterogeneity of the virus preparation without aggregation of virus capsids in the virus preparation.

A fourteenth aspect may include the features of the thirteenth aspect, and may further comprise varying a cooling profile corresponding to a manner in which the increased temperature is reduced following the respective incubation period, and determining, based on the mass spectrums, an optimum cooling profile along with the optimum ones of the temperature and the incubation period which, in combination, minimize, or at least reduce, the heterogeneity of the virus preparation without aggregation of virus capsids in the virus preparation.

A fifteenth aspect may include the features of the thirteenth aspect, and may further comprise varying a heating profile corresponding to a manner in which the temperature of the at least one of the virus preparation and the generated virus ions is increased, and determining, based on the mass spectrums, an optimum heating profile along with the optimum ones of the temperature and the incubation period which, in combination, minimize, or at least reduce, the heterogeneity of the virus preparation without aggregation of virus capsids in the virus preparation.

A sixteenth aspect may include the features of the fifteenth aspect, and may further comprise varying a cooling profile corresponding to a manner in which the increased temperature is reduced following the respective incubation period, and determining, based on the mass spectrums, an optimum cooling profile along with the optimum heating profile and the optimum ones of the temperature and the incubation period which, in combination, minimize, or at least reduce, the heterogeneity of the virus preparation without aggregation of virus capsids in the virus preparation.

A seventeenth aspect may include the features of any of the thirteenth through sixteenth aspects, wherein measuring mass-to-charge ratios and charge magnitudes of at least some of the generated virus ions at each increase of the at least one of the temperature and the incubation period is carried out using a charge detection mass spectrometer.

An eighteenth aspect may include the features of any of the thirteenth through sixteenth aspects, wherein measuring mass-to-charge ratios and charge magnitudes of at least some of the generated virus ions at each increase of the at least one of the temperature and the incubation period is carried out using a mass spectrometer.

A nineteenth aspect may include the features of any of the thirteenth through eighteenth aspects, and may further comprise determining the heterogeneity of the virus population at each increase of the at least one of the temperature and the incubation period based on mass resolution of at least one mass peak of interest in the respective mass spectrum.

A twentieth aspect may include the features of any of the thirteenth through nineteenth aspects, and may further comprise determining at each increase of the at least one of the temperature and the incubation period that aggregation has occurred if the respective mass spectrum includes discernable particles with masses greater than that of a highest mass capsid in the virus preparation, and wherein at least one of the optimum ones of the temperature and the incubation period is less than the respective temperature and incubation period of a respective mass spectrum in which aggregation has occurred.

A twenty first aspect may include the features of any of the thirteenth through twentieth aspects, and may further comprise treating other samples of the virus preparation to minimize, or at least reduce, heterogeneity thereof by heating each of the other samples of the virus preparation to the determined optimum temperature for the optimum incubation period.

A twenty second aspect may include the features of any of the thirteenth through twenty first aspects, wherein the virus preparation is a virus preparation solution, and wherein generating the virus ions comprises generating the virus ions from the virus preparation solution using an electrospray ionization source.

A twenty third aspect may include the features of any of the thirteenth through twenty second aspects, wherein sequentially increasing the at least one of the temperature and the incubation period comprises controlling a first thermal energy device coupled to the virus preparation to heat the virus preparation.

### BRIEF DESCRIPTION OF THE DRAWINGS

FIG. 1 is a simplified diagram of an embodiment of an instrument for repeatedly generating charged particles from a virus preparation and then determining and analyzing the masses of the charged particles, wherein the virus preparation and/or the charged particles is/are subjected to at least one range of differing temperature, incubation period, heating profile and/or cooling profile.

FIG. 2 is a simplified flow diagram of an embodiment of a process for controlling one or more of the thermal energy sources illustrated in FIG. 1 to subject the virus preparation and/or the charged particles to at least one range of differing temperature, incubation period, heating profile and/or cooling profile, and for then controlling the instrument to generate charged particles from the virus preparation and determine and analyze the masses of the charged particles at each combination of temperature, incubation period, heating profile and/or cooling profile to determine at least one optimum combination of temperature, incubation period, heating profile and/or cooling profile which minimizes, or at least reduces, the heterogeneity of the virus preparation without aggregation of virus capsids remaining in the preparation.

FIG. 3 is a simplified flow diagram of an embodiment of a process for treating a virus preparation in accordance with an optimum set of temperature, incubation period, heating profile and/or cooling profile, previously determined using the process illustrated in FIG. 2, for the purpose of minimizing, or at least reducing, the heterogeneity of the virus preparation without aggregation of virus capsids remaining in the preparation.

FIG. 4A is a plot of mass vs. abundance illustrating operation of the process of FIG. 2 on an example virus preparation under ambient conditions (25° C.).

FIG. 4B is a plot of mass vs. abundance illustrating operation of the process of FIG. 2 on the example virus preparation elevated to 45° C. for an example incubation period of 10 minutes.

FIG. 4C is a plot of mass vs. abundance illustrating operation of the process of FIG. 2 on the example virus preparation elevated to 50° C. for an example incubation period of 10 minutes.

FIG. 4D is a plot of mass vs. abundance illustrating operation of the process of FIG. 2 on the example virus preparation elevated to 55° C. for an example incubation period of 10 minutes.

FIG. 4E is a plot of mass vs. abundance illustrating operation of the process of FIG. 2 on the example virus preparation elevated to 60° C. for an example incubation period of 10 minutes.

FIG. 4F is a plot of mass vs. abundance illustrating operation of the process of FIG. 2 on the example virus preparation elevated to 65° C. for an example incubation period of 10 minutes.

FIG. 5A is a plot of mass vs. intensity illustrating operation of the process of FIG. 2 on another example virus preparation under ambient conditions (25° C.).

FIG. 5B is a plot of mass vs. intensity illustrating operation of the process of FIG. 2 on the example virus preparation elevated to 55° C. for an example incubation period of 20 minutes.

FIG. 5C is a plot of mass vs. intensity illustrating operation of the process of FIG. 2 on the example virus preparation elevated to 55° C. for an example incubation period of 30 minutes.

FIG. 5D is a plot of mass vs. intensity illustrating operation of the process of FIG. 2 on the example virus preparation elevated to 55° C. for an example incubation period of 40 minutes.

FIG. 5E is a plot of mass vs. intensity illustrating operation of the process of FIG. 2 on the example virus preparation elevated to 55° C. for an example incubation period of 60 minutes.

### DESCRIPTION OF THE ILLUSTRATIVE EMBODIMENTS

For the purposes of promoting an understanding of the principles of this disclosure, reference will now be made to a number of illustrative embodiments shown in the attached drawings and specific language will be used to describe the same.

This disclosure relates to apparatuses and techniques for repeatedly generating charged particles from a virus preparation and then determining and analyzing the masses of the charged particles, wherein the virus preparation and/or the charged particles is/are subjected to at least one range of differing temperature, incubation period, heating profile and/or cooling profile, for the purpose of determining at least one optimum combination of temperature, incubation period, heating profile and/or cooling profile which minimizes, or at least reduces, the heterogeneity of the preparation without aggregation of virus capsids remaining in the preparation. This disclosure also relates to apparatuses and techniques for subsequently processing a virus preparation by subjecting the virus preparation to a previously determined optimum combination of temperature, incubation period, heating profile and/or cooling profile, to produce a treated virus preparation in which the heterogeneity of the preparation is minimized, or at least reduced, without aggregation of the virus capsids remaining in the preparation. The apparatuses and techniques illustrated in the attached figures and described herein may illustratively be used to construct a library of optimum combinations of temperatures, incubation periods, heating profiles and/or cooling profiles each for processing different preparations of the virus and/or for processing preparations of different viruses for the purpose of minimizing, or at least reducing, the heterogeneity of such preparations without aggregation of the virus capsids remaining in the preparations. For purposes of this document, the term “incubation period” should be understood to mean an amount of time spent by a virus preparation and/or by charged particles generated from the virus preparation at a particular temperature. The term “aggregation” should be understood to mean adherence or attachment to one another of two or more virus capsids or capsid fragments which, will typically occur in a virus preparation at various combinations of elevated temperature and incubation period. For purposes of this disclosure, the terms “ion(s)” and “charged particle(s)” will be understood to be synonymous and may therefore be used interchangeably.

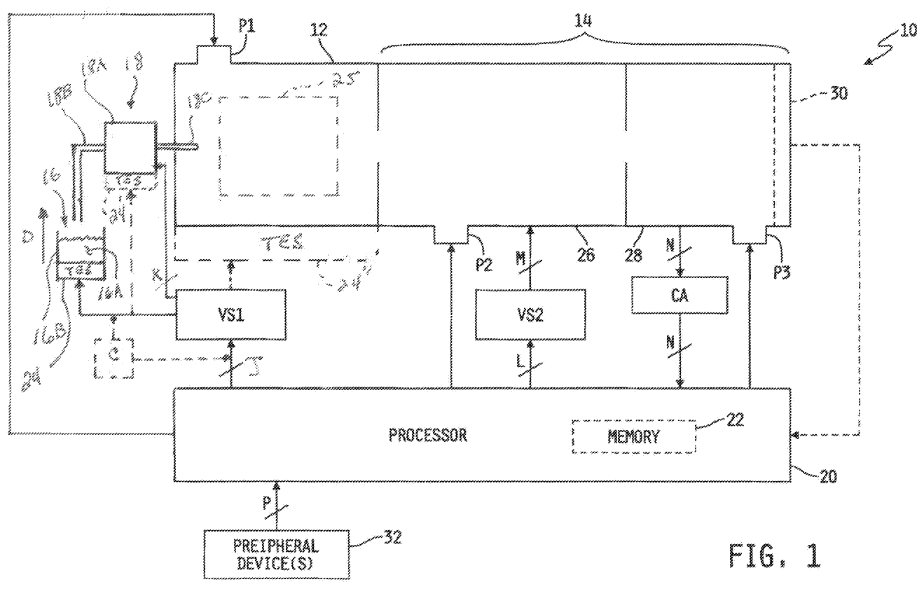

Referring now to FIG. 1, a diagram is shown of an instrument 10 for repeatedly generating charged particles from a virus preparation and then determining and analyzing the masses of the charged particles, wherein the virus preparation and/or the charged particles is/are subjected to at least one range of differing temperature, incubation period, heating profile and/or cooling profile. In the illustrated embodiment, the instrument 10 illustratively includes an ion source region 12 having an outlet coupled to an inlet of a mass spectrometer 14.

The ion source region 12 illustratively includes an ion generator 18 configured to generate ions, i.e., charged particles, from a sample 16. In the illustrated embodiment, the ion generator 18 is implemented in the form of a conventional electrospray ionization (ESI) source having a pump 18A coupled at a solution inlet to an inlet tube 18B and coupled at a solution outlet to a capillary 18C having a capillary outlet disposed in the ion source region 12 of the instrument 10. The ESI source 18 is operable in a conventional manner to draw a solution, e.g., at ambient pressure, through the inlet tube 18B into the pump 18A and to emit a fine spray or droplets of charged solution particles into the source region 12 of the instrument 10 via the outlet of the capillary 18C. In alternate embodiments, the ion generator 18 may be any conventional device or apparatus for generating ions from a sample, and may be positioned outside of the ion source region 12 or within the source region 12. As one illustrative example of the latter, which should not be considered to be limiting in any way, the structure 25 illustrated in FIG. 1 may represent the ion generator 18 in the form of a conventional matrix-assisted laser desorption ionization (MALDI) source or other conventional ion generator configured to generate ions from a sample 16 placed inside the ion source region 12. In some embodiments, the structure 25 illustrated in FIG. 1 may alternatively or additionally represent an ion inlet interface such as any of the structures disclosed in co-pending International Application No. PCT/US2019/035379, filed Jun. 4, 2019, the disclosure of which is incorporated herein by reference in its entirety. In other embodiments the structure 25 may be omitted.

The sample 16 from which the ions are generated may illustratively be any virus preparation, such as any mixture or solution of or including any type of virus, one non-limiting example of which is AAV as described above. In alternate embodiments, the sample 16 may be any mixture, solution or other form of biological and/or non-biological components. In the example illustrated in FIG. 1, the sample 16 is a virus preparation dissolved, dispersed or otherwise carried in solution 16A, e.g., in a container 16B, although in other embodiments the sample 16 may not be in or part of a solution. In the example embodiment illustrated in FIG. 1, the container 16B is shown displaced downwardly away from the inlet tube 18B of the ESI source 18, and it will be understood that the container 16B is movable upwardly in the direction D such that the inlet tube 18B will be in fluid communication with the sample solution 16A.

In the illustrated embodiment, a voltage source VS1 is electrically connected to a processor 20 via a number, J, of signal paths, where J may be any positive integer, and is further electrically connected to the ion generator 18 via a number, K, of signal paths, where K may likewise be any positive integer. In some embodiments, the voltage source VS1 may be implemented in the form of a single voltage source, and in other embodiments the voltage source VS1 may include any number of separate voltage sources. In some embodiments, the voltage source VS1 may be configured or controlled to produce and supply one or more time-invariant (i.e., DC) voltages of selectable magnitude. Alternatively or additionally, the voltage source VS1 may be configured or controlled to produce and supply one or more switchable time-invariant voltages, i.e., one or more switchable DC voltages. Alternatively or additionally, the voltage source VS1 may be configured or controllable to produce and supply one or more time-varying signals of selectable shape, duty cycle, peak magnitude and/or frequency.

The processor 20 is illustratively conventional and may include a single processing circuit or multiple processing circuits. The processor 20 illustratively includes or is coupled to a memory 22 having instructions stored therein which, when executed by the processor 20, cause the processor 20 to control the voltage source VS1 to produce one or more output voltages for selectively controlling operation of the ion generator 18. In some embodiments, the processor 20 may be implemented in the form of one or more conventional microprocessors or controllers, and in such embodiments the memory 22 may be implemented in the form of one or more conventional memory units having stored therein the instructions in a form of one or more microprocessor-executable instructions or instruction sets. In other embodiments, the processor 20 may be alternatively or additionally implemented in the form of a field programmable gate array (FPGA) or similar circuitry, and in such embodiments the memory 22 may be implemented in the form of programmable logic blocks contained in and/or outside of the FPGA within which the instructions may be programmed and stored. In still other embodiments, the processor 20 and/or memory 22 may be implemented in the form of one or more application specific integrated circuits (ASICs). Those skilled in the art will recognize other forms in which the processor 20 and/or the memory 22 may be implemented, and it will be understood that any such other forms of implementation are contemplated by, and are intended to fall within, this disclosure. In some alternative embodiments, the voltage source VS1 may itself be programmable to selectively produce one or more constant and/or time-varying output voltages.

In the illustrated embodiment, the voltage source VS1 is illustratively configured to be responsive to control signals produced by the processor 20 to produce one or more voltages to cause the ion generator 18 to generate ions from the sample 16 in a conventional manner. In some embodiments, the sample 16 is positioned outside of the ion source region 12, as illustrated in FIG. 1, and in other embodiments the ion source 18 may be positioned within the ion source region 12. In the illustrated embodiment, the electrospray ionization (ESI) source 18 is configured to be responsive to one or more voltages supplied by VS1 to generate ions from the sample 16 in the form of a fine mist of charged droplets. It will be understood that ESI and MALDI, as described hereinabove, represent only two examples of myriad conventional ion generators, and that the ion generator 18 may be or include any such conventional device or apparatus for generating ions from a sample whether or not in solution.

At least one thermal energy source is configured to selectively thermally energize, i.e., transfer thermal energy to, the sample 16 and/or to the ion generator 18 and/or to the charged particles within the ion source region 12. In the illustrated embodiment, for example, a thermal energy source 24 is shown operatively coupled to the container 16B carrying the solution 16A containing a virus preparation, and in this embodiment the thermal energy source 24 is configured to transfer thermal energy to the virus preparation solution 16A via the container 16B. Alternatively or additionally, a thermal energy source 24′ may be operatively coupled to the ion generator 18. In some such embodiments, the thermal energy source 24′ may be coupled to the pump 18A and/or to the inlet tube 18B, and in such embodiments the thermal energy source 24′ is configured to transfer thermal energy to the virus preparation solution 16A via the pump 18A and/or the tube 18B, e.g., prior to ionization of the solution 16A. In other such embodiments, the thermal energy source 24′ may be coupled to the capillary 18C, and in such embodiments the thermal energy source 24′ is configured to transfer thermal energy to the solution 16A within the capillary 18C and/or to the charged particles exiting the capillary 18C. Alternatively or additionally still, a thermal energy source 24″ may be operatively coupled to the ion source region 12 of the instrument 10, and in such embodiments the thermal energy source 24″ is configured to transfer thermal energy to the charged particles within the ion source region 12, i.e., to the charged particles exiting the ion generator 18 and prior to entrance of the charged particles into the mass spectrometer 14.

In some embodiments, the thermal energy produced by the thermal energy source 24, 24′, 24″ may be in the form of heat transferred from the source 24, 24′, 24″ to the sample 16, ion generator 18 and/or charged particles, and in other embodiments the thermal energy may be in the form of heat transferred from the sample 16, ion generator 18 and/or charged particles to the source 24, 24′, 24″, i.e., cooling of the sample particles. In some embodiments, the source 24′, 24′, 24″ may include both heating and cooling capabilities so that the sample temperature may be swept through ambient temperature from warmer to cooler or from cooler to warmer, or may be swept from any of cold to colder, colder to less cold, cold or cool to warm or hot, warm or hot to cool or cold, warm to warmer, warmer to less warm, warm to hot, hot to warm, etc. Example heat sources 24, 24′, 24″ may include, but are not limited to, conventional solution heaters and heating units, one or more sources of radiation, e.g., infrared, laser, microwave or other, at any radiation frequency, one or more heated gasses or other fluid(s) or the like, and example cooling sources 24, 24′, 24″ may include, but are not limited to, conventional solution chillers, one or more chilled gasses or other fluid(s), or the like. Some examples of the thermal energy source 24″ and operation thereof for heating charged particles are disclosed in co-pending International Application No. PCT/US2018/064005, filed Dec. 5, 2018, the disclosure of which is incorporated herein by reference in its entirety. Those skilled in the art will recognize other structures and/or techniques for controlling the temperature of the virus preparation 16 by heating or cooling prior to or after generating charged particles therefrom, and it will be understood that any such other structures and/or techniques are intended to fall within the scope of this disclosure.

In some embodiments, as illustrated by example in FIG. 1, the thermal energy source 24, 24′, 24″ is electrically connected to the voltage source VS1, and the voltage source VS1 is configured to be responsive to one or more control signals produced by the processor 20 to produce one or more corresponding voltages to control thermal energy produced by the thermal energy source 24, 24′, 24″. In alternate embodiments, the thermal energy source 24, 24′, 24″ may be configured to be responsive to control signals produced by the processor 20 to selectively produce thermal energy, and in such embodiments the thermal energy source 24, 24′, 24″ may be electrically connected directly, or via conventional circuitry, to the processor 20. In some embodiments which include the thermal energy source 24 and/or the thermal energy source 24′, the voltage/current supplied thereto by the voltage source VS1 or the thermal energy source 24, 24′ itself may not be controlled by the processor 20 but rather by a separate, conventional control circuit C as illustrated by dashed-line representation in FIG. 1. In some embodiments, the thermal energy source 24, 24′, 24″ may be a conventional, manually-controlled thermal energy source, e.g., such as a manually controlled heater and/or ice bath, and in such embodiments operation of the thermal energy source 24, 24′, 24″ will not be controlled by the processor 20 or the control circuit C, but will instead be controlled manually, e.g., by manually-controlling the thermal energy source, monitoring temperature manually, e.g., via a conventional thermometer or temperature sensor and/or monitoring incubation period manually, e.g., via a conventional timer, timepiece or similar device. In any case, the thermal energy source 24, 24′, 24″ may be implemented in the form of one or more conventional heaters or heating elements and/or one or more conventional coolers or cooling elements.

In embodiments in which the thermal energy source 24, 24′, 24″ is/are controlled by the processor 20 or by a control circuit C, the thermal energy source 24, 24′, 24″ is responsive to one or more voltages produced by the voltage source VS1 and/or to one or more control signals produced by the processor 20 or the control circuit C to control the temperature of the sample 16, the temperature of the ion generator 18 and/or the temperature of charged particles within the ion source region 12 as well as the incubation period, i.e., the time duration at which the thermal energy source 24, 24′, 24″ is controlled to any specific temperature.

In some embodiments, the thermal energy source 24, 24′, 24″ is configured to be responsive to the one or more voltages produced by the voltage source VS1 to achieve a target, elevated temperature as quickly as practicable given the physical limitations of the thermal energy source 24, 24′, 24″. In alternate embodiments, the thermal energy source 24, 24′, 24″ may be configured to be programmed or to be responsive to control signals produced by the processor 20 or control circuit C to achieve the target, elevated temperature according to any of a plurality of different heating profiles. Examples of such heating profiles may include, but are not limited to, a linearly increasing, e.g., ramped, temperature profile, a non-linearly or piece-wise linearly increasing temperature profile or a combination thereof. In some such embodiments, the duration of the heating profile, i.e., between the present temperature and the target, elevated temperature, may also be controlled by the processor 20 or control circuit C.

In some embodiments, the thermal energy source 24, 24′, 24″ is configured to be responsive to the one or more voltages produced by the voltage source VS1 to achieve a target, reduced temperature as quickly as practicable given the physical limitations of the thermal energy source 24, 24′, 24″. As one example, the thermal energy source 24, 24′, 24″ may be configured to achieve the target, reduced temperature simply by turning off, or turning down, the thermal energy source 24, 24′, 24″, in which case the target, reduced temperature will be achieved over a time duration in which the thermal energy source 24, 24′, 24″ and the sample 16, ion generator 18 and/or ion source region 12 together cool to the target, reduced temperature. In alternate embodiments, the thermal energy source 24, 24′, 24″ may be configured to be programmed or to be responsive to control signals produced by the processor 20 or control circuit C to achieve the target, reduced temperature according to any of a plurality of different cooling profiles. Examples of such cooling profiles may include, but are not limited to, a linearly decreasing, e.g., ramped, temperature profile, a non-linearly or piece-wise linearly decreasing temperature profile or a combination thereof. In some such embodiments, the duration of the cooling profile, i.e., between the present temperature and the target, reduced temperature, may also be controlled by the processor 20 or control circuit C.

In some embodiments, the sample 16, the ion source 18 and/or the charged particles in the ion source region 12 is/are allowed to cool, or is/are actively cooled as just described, such that analysis by the mass spectrometer 14 is carried out on charged particles at or near ambient temperature. In other embodiments, the sample 16, the ion source 18 and/or the charged particles in the ion source 12 may be cooled to below ambient temperature such that analysis by the mass spectrometer 14 is carried out on charged particles cooled to a temperature below ambient. In still other embodiments, the sample 16, the ion source 18 and/or the charged particles in the ion source 12 is/are heated to one or more elevated temperatures for one or more incubation periods as just described, but are then not substantially cooled such that analysis by the mass spectrometer 14 is carried out on charged particles heated to one or more elevated temperatures each for one or more incubation periods.

The mass spectrometer 14 illustratively includes two sections coupled together; an ion processing region 26 and an ion detection region 28. A second voltage source VS2 is electrically connected to the processor 20 via a number, L, of signal paths, where L may be any positive integer, and is further electrically connected to the ion processing region 26 via a number, M, of signal paths, where M may likewise be any positive integer. In some embodiments, the voltage source VS2 may be implemented in the form of a single voltage source, and in other embodiments the voltage source VS2 may include any number of separate voltage sources. In some embodiments, the voltage source VS2 may be configured or controlled to produce and supply one or more time-invariant (i.e., DC) voltages of selectable magnitude. Alternatively or additionally, the voltage source VS2 may be configured or controlled to produce and supply one or more switchable time-invariant voltages, i.e., one or more switchable DC voltages. Alternatively or additionally, the voltage source VS2 may be configured or controllable to produce and supply one or more time-varying signals of selectable shape, duty cycle, peak magnitude and/or frequency. As one specific example of the latter embodiment, which should not be considered to be limiting in any way, the voltage source VS2 may be configured or controllable to produce and supply one or more time-varying voltages in the form of one or more sinusoidal (or other shaped) voltages in the radio frequency (RF) range.

In some embodiments, the mass spectrometer 14 is configured to simultaneously measure both mass-to-charge ratios and charge magnitudes of charged particles generated by the ion generator 18, such that the processor 20 can then determine ion mass based on these measurements. In such embodiments, the ion detection region 28 is electrically connected to input(s) of each of a number, N, of charge detection amplifiers CA, where N may be any positive integer, and output(s) of the number, N, of charge detection amplifiers CA is/are electrically connected to the processor 20 as shown in FIG. 1. The charge detection amplifier(s) CA is/are each illustratively conventional and responsive to charges induced by charged particles on one or more respective charge detectors disposed in the charge detection region 28 to produce corresponding charge detection signals at the output thereof, and to supply the charge detection signals to the processor 20.

In one embodiment in which the mass spectrometer 14 is provided in the form of a mass spectrometer configured to simultaneously measure both mass-to-charge ratios and charge magnitudes of charged particles generated by the ion generator 18, the mass spectrometer 14 may be implemented in the form of a charge detection mass spectrometer (CDMS), wherein the ion processing region 26 is or includes a conventional mass spectrometer or mass analyzer and the ion detection region 28 illustratively includes one or more corresponding CDMS charge detectors. In some embodiments, the one or more CDMS charge detectors may be provided in the form of one or more electrostatic linear ion traps (ELITs), and in other embodiments the one or more CDMS charge detectors may be provided in the form of at least one orbitrap. In some embodiments, the CDMS charge detector(s) may include at least one ELIT and at least one orbitrap. CDMS is illustratively a single-particle technique typically operable to measure mass-to-charge ratios and charge magnitude values of single ions, although some CDMS detectors have been designed and/or operated to measure mass-to-charge ratios and charge magnitudes of more than one charged particle at a time. Some examples of CDMS instruments and/or techniques, and of CDMS charge detectors and/or techniques, which may be implemented in or as the mass spectrometer 14 of FIG. 1 are disclosed in co-pending International Application Nos. PCT/US2019/013251, PCT/US2019/013274, PCT/US2019/013277, PCT/US2019/013278, PCT/US2019/013280, PCT/US2019/013283, PCT/US2019/013284 and PCT/US2019/013285, all filed Jan. 11, 2019, and the disclosures of which are all incorporated herein by reference in their entireties.

In another embodiment in which the mass spectrometer 14 is provided in the form of a mass spectrometer configured to simultaneously measure both mass-to-charge ratios and charge magnitudes of charged particles generated by the ion generator 18, the mass spectrometer 14 may be implemented in the form of a mass spectrometer configured to measure mass-to-charge ratios of charged particles and further configured to simultaneously measure charge magnitudes of the charged particles. In such embodiments, the ion processing region 26 is or includes an ion acceleration region and/or a scanning mass-to-charge ratio filter, and the ion detection region 28 illustratively includes a charge detector array disposed in an electric field-free drift region or drift tube. In such embodiments, a conventional ion detector 30, e.g., a conventional microchannel plate detector or other conventional ion detector, is positioned at the outlet end of the drift region or drift tube and is electrically connected to the processor as illustrated by dashed-line representation in FIG. 1. Some example embodiments of such a mass spectrometer are disclosed in co-pending International Application No. PCT/US2020/065301, filed Dec. 16, 2020, the disclosure of which is incorporated herein by reference in its entirety.

Regardless of the particular form in which the mass spectrometer 14 is provided, the various sections of the instrument 10 are controlled to sub-atmospheric pressure for operation thereof as is conventional. In the illustrated embodiment, for example, a so-called vacuum pump P1 is operatively coupled to the ion source region 12, another vacuum pump P2 is operatively coupled to the ion processing region 26 of the mass spectrometer 14 and yet another vacuum pump P2 is operatively coupled to the ion detection region 28 of the mass spectrometer. In the illustrated embodiment, each of the pumps P1, P2 and P3 is operatively coupled to the processor 20 such that the processor 20 is configured to control operation of each of the pumps P1, P2 and P3 and therefore independently control the pressures in each of the three respective regions 12, 26 and 28. In alternate embodiments, one or more of the pumps P1, P2 and/or P3 may be manually controlled. In still other embodiments, more or fewer pumps may be implemented to control the pressure in more or fewer respective portions of the instrument 10. The pressures in the regions 12, 26 and 28 are illustratively set in a conventional manner to provide for positive gas flow in the direction of the region 28.

The instrument 10 further illustratively includes one or more peripheral devices 32 operatively coupled to the processor 20 via a number, P, of signal paths, wherein P may be any positive integer. The peripheral device(s) 32 may be or include any one or combination of conventional peripheral devices including, for example, but not limited to, one or more monitors, keyboards, key pads, point-and-click devices, printers, graphical displays, etc.

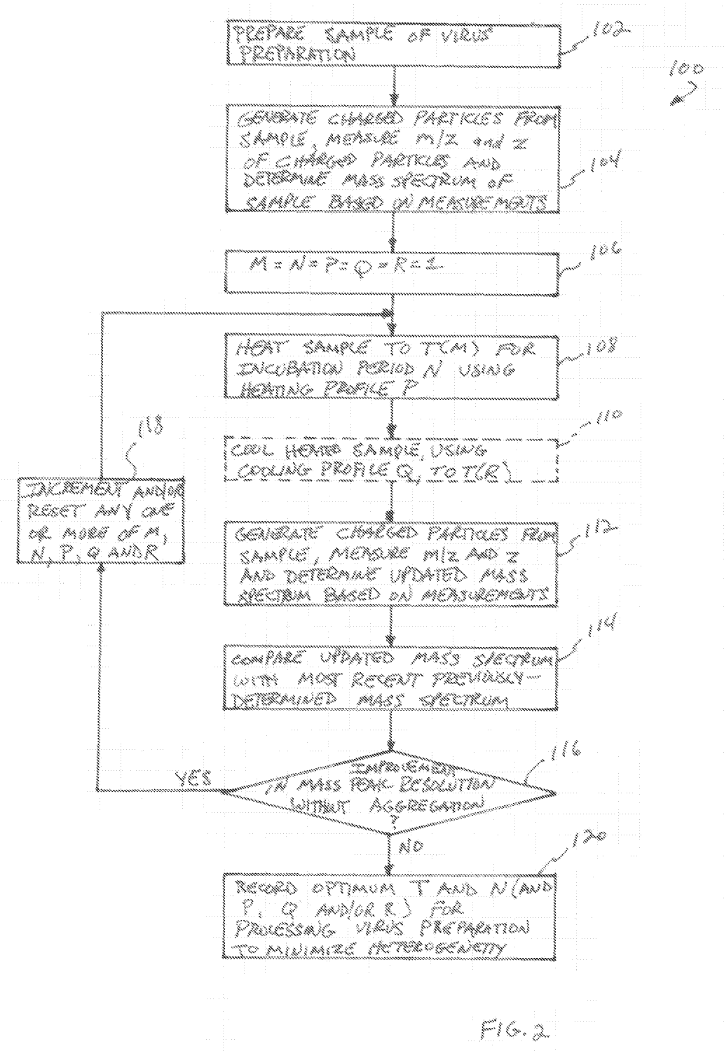

Referring now to FIG. 2, a simplified flowchart is shown depicting an example process 100 for controlling the thermal energy source(s) 24, 24′, 24″ to subject the virus preparation 16 and/or the charged virus preparation particles generated by the ion generator 18 to at least one range of differing temperature, incubation period, heating profile and/or cooling profile, and for also controlling the instrument 10 to generate charged particles from the virus preparation and determine and analyze the masses of the charged particles at each combination of temperature, incubation period, heating profile and/or cooling profile to determine at least one optimum combination of temperature, incubation period, heating profile and/or cooling profile which minimizes, or at least reduces, the heterogeneity of the virus preparation without aggregation of virus capsids remaining in the preparation. Some of the steps of the process 100 are illustratively provided in the form of instructions stored in the memory 22 and executable by the processor 20 to carry out the corresponding functions described below, and others of the steps may be carried out manually or by the control circuit C illustrated in FIG. 1.

The process 100 begins at step 102 where a sample 16 of a virus preparation is prepared or obtained. As described above, the virus preparation 16 may contain any type of virus or combination of viruses without limitation. For purposes of the following description of the process 100, the virus preparation 16 is illustratively a virus preparation in solution so as to be in a form from which the ESI source 18 depicted in FIG. 1 can generate charged particles in the form of a fine mist or droplets as described above. It will be understood, however, that the virus preparation 16 in other embodiments may be provided in non-solution form and/or that the ion generator in other embodiments may be another conventional ion generator, examples of which are described above.

The process 100 advances from step 102 to step 104 where the processor 20 is operable, pursuant to execution of corresponding instructions stored in the memory 22, to control the ion generator 18 to generate charged particles from the virus preparation 16, wherein the charged particles are directed by the instrument 10 through the ion source region 12 into the mass spectrometer 14, e.g., via a pressure differential between the atmospheric pressure of the ESI source 18 and the vacuum conditions of the ion source region 12 and/or via a pressure differential between the vacuum conditions of the ion source region 12 and the lower vacuum conditions of the mass spectrometer 14 and/or via an inlet interface 25 in embodiments which include the interface 25. The processor 20 is further operable at step 104 to control the mass spectrometer 14 to measure the mass-to-charge ratios and charge magnitudes of the generated charged particles as described above, to then compute the masses of the charged particles based on the mass-to-charge ratio and charge magnitude measurements and generate a mass spectrum of the charged particle masses. At step 104, the virus preparation is illustratively at ambient temperature, e.g., 25° C., and has not yet been subject to elevated temperature treatment, and the measurements taken by the instrument 10 at step 104 are likewise at ambient temperature. In alternate embodiments, the virus preparation 16 and/or the measurements taken by the instrument 10 at step 104 may be greater than or less than ambient temperature.

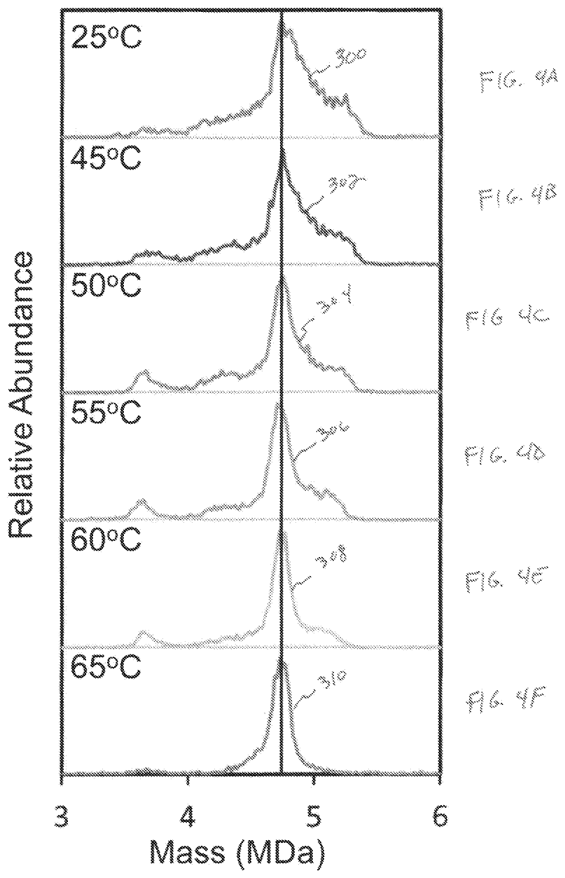

An example of a mass spectrum 300 generated at step 104 is illustrated in FIG. 4A. The example mass spectrum 300 is represented in FIG. 4A as a plot of abundance vs. mass of a virus preparation solution 16 containing AAV8 with EF1a-GFP genome and has a broad mass peak at approximately 4.6 MDa. The temperature of the virus preparation 16 was 25° C. and the mass spectrum 300 was likewise measured by the instrument 10 at 25° C. In some alternate embodiments, the mass spectrum 300 may take the form of measured ion intensity vs. mass, and in other alternate embodiments the mass spectrum 300 may be represented in the form of particle charge vs. particle mass (i.e., a scatter plot).

Following step 104, the process 100 advances to step 106 where a number of counters, e.g., M, N, P, Q and R, are illustratively set, e.g., to a starting value, such as 1. Thereafter at step 108, the virus preparation 16, the ion generator 18 and/or the charged particles resident within the ion source region 12 is/are illustratively heated to an elevated temperature T(M), i.e., T1 at the first execution of step 108, for an incubation period N, e.g., incubation period 1 at the first execution of step 108, using a heating profile P, e.g., heating profile 1 at the first execution of step 108. The temperature change(s), i.e., the temperature step size(s), between the measurements taken under ambient conditions, e.g., 25° C., at step 104 and the temperature T(1), as well as those between each T(M) at each execution of step 108, may have any integer or non-integer value, and may or may not have the same value at each execution of step 108. At the first execution of step 1, T(1) is illustratively greater than the temperature conditions of step 104. At subsequent executions of step 108, T(M) may or may not change relative to the previous execution of step 108, and any changes in T(M) in any such subsequent executions of step 108 may or may not be uniform or constant. The virus preparation 16, the ion generator 18 and/or the charged particles resident within the ion source region 12 may be heated at step 108 using any one or combination of the various devices, apparatuses and/or techniques described above with respect to FIG. 1. In some embodiments, for example, the processor 20 may be operable to execute instructions stored in the memory 22 to cause the processor 20 to control the voltage source V1 to control the thermal energy source 24 to heat the virus preparation 16 to the temperature T(M) for the corresponding incubation period N using the heating profile P. Alternatively or additionally, the processor 20 may be operable to execute instructions stored in the memory 22 to cause the processor 20 to control the voltage source V1 to control the thermal energy source 24′ to heat the ion generator 18 in a manner that heats the virus preparation 16 contained in any part thereof to the temperature T(M) for the corresponding incubation period N using the heating profile P and/or to cause the processor 20 to control the voltage source V1 to control the thermal energy source 24″ to heat the charged particles emitted by the ion source 18 into the ion source region 12 to the temperature T(M) for the corresponding incubation period N using the heating profile P. Alternatively still, the control circuit C may be programmed to control any one or combination of the thermal energy sources 24, 24′, 24″ alternatively to, or in addition to, control thereof by the processor 20. In still other embodiments, the temperature of the virus preparation 16 and/or of the ion generator 18 and/or of the ion source region 12 may be manually controlled to the temperature T(M) for the corresponding incubation period N using the heating profile P.

The incubation period(s), i.e., the time duration(s) spent by the virus preparation 16, the ion generator 18 and/or the charged particles resident within the ion source region 12 at the temperature T(M) set at each execution of step 108, may have any value of any one or combination of days, hours, minutes, seconds and/or fractions of seconds, and the incubation period at any execution of step 108 may or may not have the same duration as the incubation step at any other execution of step 108. The heating profile(s), i.e., the time duration and/or manner in which the temperature(s) of the virus preparation 16, the ion generator 18, the charged particles exiting the ion generator 18 and/or the charged particles resident within the ion source region 12 is/are increased at step 108 to a temperature greater than that at step 104 or greater than that of a previous execution of step 108, may be or have any desired heating profile, some non-limiting examples of which are described above, and the heating profile used at any increase in the temperature T(M) at any execution of step 108 may or may not be the same as that used at any other execution of step 108.

In some embodiments of the process 100, the virus preparation 16, the ion generator 18 and/or the charged particles resident within the ion source region 12 is/are cooled, following step 108 and prior to processing by the instrument 10, to a temperature that is less than that at step 108. In such embodiments, the process 100 illustratively includes step 110 to which the process 100 advances following execution of step 108, wherein the virus preparation 16, the ion generator 18 and/or the charged particles resident within the ion source region 12 is/are cooled to a reduced temperature, T(R), using a cooling profile Q, e.g., heating profile 1 at the first execution of step 110. The cooling profile(s), i.e., the time duration and/or manner in which the temperature(s) of the virus preparation 16, the ion generator 18, the charged particles exiting the ion generator 18 and/or the charged particles resident within the ion source region 12 is/are reduced at step 110 to a temperature less than that at step 108, may be or have any desired cooling profile, some non-limiting examples of which are described above, and the cooling profile used at any decrease in the temperature T(Q) at any execution of step 110 may or may not be the same as that used at any other execution of step 110. The virus preparation 16, the ion generator 18 and/or the charged particles resident within the ion source region 12 may be cooled at step 110 using any one or combination of the various devices, apparatuses and/or techniques described above with respect to FIG. 1.

In some embodiments, the virus preparation 16, the ion generator 18 and/or the charged particles resident within the ion source region 12 is/are cooled to ambient temperature(s), e.g., 25° C., after each execution of step 108 in which the virus preparation 16, the ion generator 18 and/or the charged particles resident within the ion source region 12 is/are heated to an elevated temperature above ambient temperature, such that the measurements performed by the mass spectrometer 14 in any case are carried out on charged particles at ambient temperature, e.g., 25° C. In one example such embodiment, each execution of step 108 is carried out by heating only the virus preparation 16 to an elevated temperature T(M) for an incubation period N using a heating profile P, and then the virus preparation 16 is cooled to ambient temperature thereafter at step 110 such that the virus preparation is not acted upon by the instrument 10 until the heating, incubation and cooling steps are complete. In alternate embodiments, step 110 may be omitted such that charged particles resulting from heating of the virus preparation 16, the ion generator 18 and/or the charged particles resident within the ion source region 12 at each execution step 108 to T(M) for a corresponding incubation period is/are measured by the instrument 10 at or near the same temperature(s) T(M).

Following step 110, in embodiments which include step 110 and otherwise following step 108, the process 100 advances to step 112 where the processor 20 is again operable, pursuant to execution of corresponding instructions stored in the memory 22, to control the ion generator 18 to generate charged particles from the virus preparation 16, wherein the charged particles are directed by the instrument 10 through the ion source region 12 into the mass spectrometer 14, to control the mass spectrometer 14 to measure the mass-to-charge ratios and charge magnitudes of the generated charged particles as described above, and to then compute the masses of the charged particles based on the mass-to-charge ratio and charge magnitude measurements and generate an updated mass spectrum of the charged particle masses. In some embodiments, as described above, the virus preparation 16 is illustratively at ambient temperature, e.g., 25° C., and the measurements taken by the instrument 10 at step 112 are likewise at ambient temperature, although in alternate embodiments, the virus preparation 16 and/or the measurements taken by the instrument 10 at step 112 may be greater than or less than ambient temperature, as also described above.

Following step 112, the process 100 advances to steps 114 and 116 where the updated mass spectrum determined at the most recent execution of step 112 is compared to the most recent previously determined mass spectrum, e.g., to the mass spectrum determined at step 104 during the first execution of step 114 and otherwise to the mass spectrum determined at the previous execution of step 114, to determine whether the updated mass spectrum indicates an improvement is mass peak resolution without aggregation of virus capsids. In some embodiments, steps 114 and 116 are executed by the processor 20, and in other embodiments either or both of the steps 114 and 116 may be carried out manually, i.e., by visually comparing the updated and previous mass spectrums. In either case, mass peak widths can be determined via the processor 20 or visually in a conventional manner.

Aggregation may likewise be determined via the processor 20 or visually. For example, when two or more virus capsids or capsid fragments adhere or attach to one another during aggregation, which will generally occur at various combinations of sufficiently high temperatures and incubation periods, the adhered or attached capsids will generally result in charged particles with higher mass and higher charge than non-aggregated capsids. Accordingly, the onset of aggregation may be detected, either visually or automatically by the processor 20, by determining whether the updated mass spectrum exhibits increased mass and/or charge values.

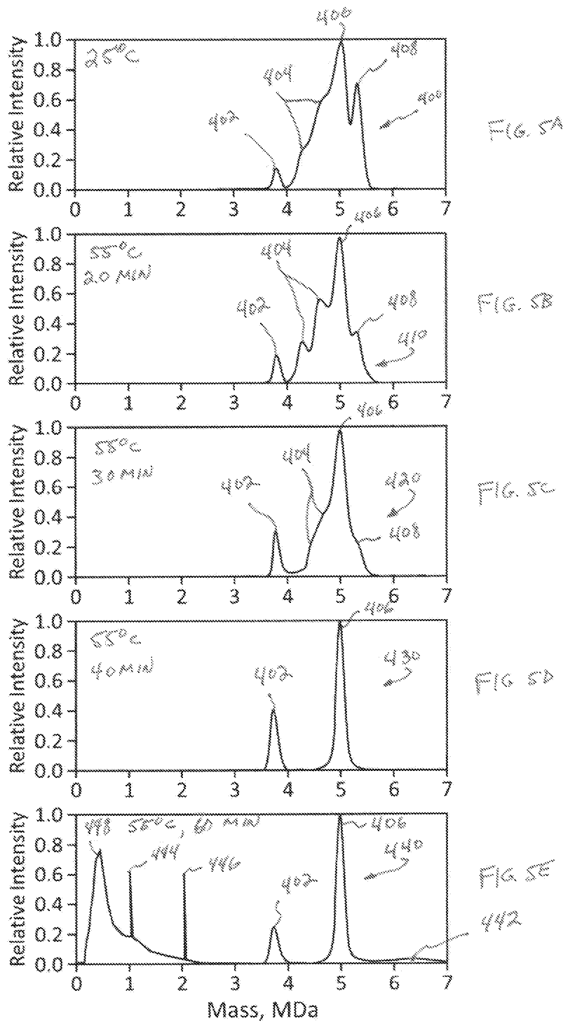

In any case, if, at step 116, the comparison made at step 114 indicates that the updated mass spectrum exhibits an improvement in mass peak resolution without aggregation, the process 100 advances to step 118 where one or more of the counters, M, N, P, Q and/or R is incremented and/or reset before looping back to step 108. As described above in detail, one or more of the temperature of the virus preparation 16, the temperature of one or more components of the ion generator 18, the charged particle temperature within the ion source region 12, the incubation period, the heating profile and the cooling profile may or may not be changed at each execution of step 118. Two different examples will be described below with respect to FIGS. 4A-4F and FIGS. 5A-5E.

If, at step 116, the comparison made at step 114 indicates that the updated mass spectrum does not exhibit an improvement in mass peak resolution or exhibits a detectable amount of aggregation, the process advances to step 120 where the variable values which generated the most recent previous mass spectrum are recorded, e.g., stored in the memory 22, as at least one optimum combination of temperature, incubation period, heating profile and, in some embodiments cooling profile, conditions for treating like virus preparations for the purpose of minimizing, or at least reducing, the heterogeneity of the preparation without aggregation of virus capsids remaining in the preparation. It will be understood that there may be other combinations of temperature, incubation period, heating profile and, in some embodiments cooling profile, conditions for treating the virus preparation which also minimize, or at least reduce, the heterogeneity of the preparation without aggregation of the virus capsids remaining in the preparation, and such alternate optimum combinations of temperature, incubation period, heating profile and, in some embodiments cooling profile, conditions, resulting from execution of the process 100 using other values of one or more of the variables, may also be recorded.

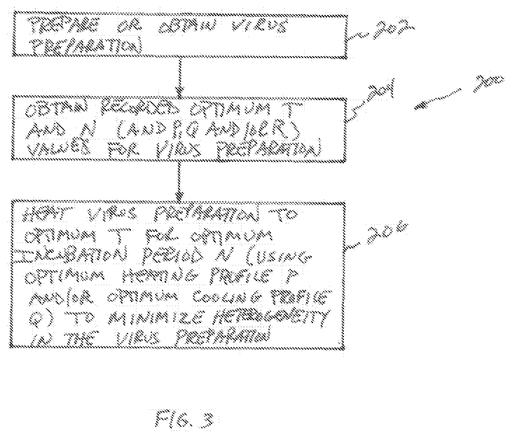

As described briefly above, the process 100 may be used to construct a library of optimum combinations of temperatures, incubation periods, heating profiles and/or cooling profiles each for processing the same preparations of a virus, different preparations of the virus and/or preparations of different viruses for the purpose of minimizing, or at least reducing, the heterogeneity of such preparations without aggregation of the virus capsids remaining in the preparations. Following recordation of at least one optimum combination of temperature, incubation period, heating profile and/or cooling profile, e.g., resulting from execution of the process 100 as just described, another process may be executed to apply the optimum combination of conditions to a like virus preparation that is as yet untreated. Referring now to FIG. 3, a simplified flow diagram of an example of such a process 200 is shown. The process 200 illustratively begins at step 202 where a virus preparation is prepared or obtained. The virus preparation may be prepared or obtained in any form, e.g., mixture, solution, bulk form, etc., and may contain any type of virus or combination of viruses without limitation. Thereafter at step 204, a previously-recorded optimum combination of temperature and incubation period, and in some embodiments heating profile and/or cooling profile) is obtained. In some embodiments, more than one optimum combination may have been previously recorded, and in such embodiments one of such a plurality of optimum combinations may be selected manually or automatically. Thereafter at step 206, the virus preparation is heated to the selected optimum temperature for the corresponding optimum incubation period. In some embodiments, the selected optimum combination may include an optimum heating profile, and in such embodiments the virus preparation may be heated at step 206 to the optimum temperature using the optimum heating profile. In some embodiments, the selected optimum combination may alternatively or additionally include an optimum cooling profile, and in such embodiments the virus preparation may be heated at step 206 to the optimum temperature for the optimum incubation period followed by cooling the virus preparation using the optimum cooling profile. In any case, following step 206, the treated virus preparation will have minimized, or at least reduced, heterogeneity without aggregation of the remaining virus capsids.

### EXAMPLES

### Example 1

Referring now to FIGS. 4A-4F, an example is shown of steps 102-118 of the process 100 illustrated in FIG. 2. As described above, FIG. 4A depicts an example of a mass spectrum 300, generated at step 104 of the process 100, in the form of a plot of abundance vs. mass of a previously untreated (by the process 100) virus preparation solution 16 containing AAV8 with EF1a-GFP genome. The temperature of the virus preparation 16 was 25° C. and the mass spectrum 300 was likewise measured by the instrument 10 at 25° C. As illustrated by example in FIG. 4A, the mass spectrum 300 has a broad mass peak at approximately 4.6 MDa.

FIG. 4B depicts another mass spectrum 302 resulting from execution of step 108 of the process 100 in which the temperature of the virus preparation 16 was increased, by controlling a conventional heating coil 24 coupled to the virus preparation to elevate the temperature of the virus preparation 16 as quickly as possible, to 45° C., and in which the temperature of the virus preparation 16 was then maintained at 45° C. for an incubation period of 15 minutes. In the illustrated example, the process 100 included step 110 in which, following expiration of the incubation period, the virus preparation 16 was removed from the heating coil, cooled on ice for 1 minute and then allowed to warm naturally to 25° C. After cooling to 25° C., step 112 was carried on the cooled virus preparation 16 with the instrument 10 likewise operating at 25° C. Comparing the mass spectrum 302 to the mass spectrum 300 at step 114, it is clear from FIGS. 4A and 4B that the mass spectrum 302 exhibits an improvement in mass peak resolution over that of the mass spectrum 300. Moreover, as the resulting mass spectrum 302 does not appear to exhibit any peaks higher in mass than that of the sole mass peak depicted in the mass spectrum 300, aggregation does not appear to be present in the mass spectrum 302. As such, step 116 advances to step 118 where, in this case, only the temperature value is changed by increasing it by 5° C.

The process steps 108-118 just described are repeated four additional times to subject the virus preparation 16 to 50° C., 55° C., 60° C. and 65° C. respectively, each for incubation periods of 15 minutes. The resulting mass spectra 304, 306, 308, 310 respectively illustrated in FIGS. 4C-4F each exhibit an improvement in mass peak resolution over that of the previously determined mass spectrum with no discernable aggregation. The example illustrated in FIGS. 4A-4F was not continued past 65° C., and an onset of aggregation was therefore not observed in this example. Accordingly, it cannot be discerned from FIGS. 4A-4F whether any additional improvements in mass peak resolution could be realized by continuing the process 100, and so it likewise cannot be discerned from FIGS. 4A-4F whether an incubation time for the example virus preparation 16 of 15 minutes at 65° C. represents an optimum combination of temperature and incubation period which minimizes heterogeneity of the virus preparation 16 without aggregation of the remaining virus capsids. It can, however, be concluded from FIGS. 4A-4F that an incubation time for the example virus preparation 16 of 15 minutes at 65° C. substantially reduces the heterogeneity of the virus preparation 16 without aggregation of the remaining virus capsids.

### Example 2

Referring now to FIGS. 5A-4E, another example is shown of the process 100 illustrated in FIG. 2 including step 120. In the illustrated example, FIG. 5A depicts an example of a mass spectrum 400, generated at step 104 of the process 100, in the form of a plot of relative ion intensity vs. mass of a previously untreated (by the process 100) and simulated virus preparation solution 16 representative of a virus preparation that may contain, for example, AAV. The temperature of the simulated virus preparation 16 was 25° C. and the simulated measurements taken by the instrument 10 to generate the mass spectrum 400 were likewise at 25° C. As illustrated by example in FIG. 5A, the mass spectrum 400 has a number of peaks each of which correspond to different contents of the virus capsids. The mass peak 402 at approximately 3.8 MDa, for example, is attributable to empty capsids, i.e., those that contain no genome or partial genome, the mass peaks 404 at approximately 4.3 MDa and 4.6 MDa are attributable to partial capsids, i.e., those that contain partial genomes or partial genomes, the mass peak 406 at approximately 5 MDa is attributable to full capsids, i.e., those that each contain a single genome, and the mass peak 408 at approximately 5.2 MDa is attributable to over-packaged capsids, i.e., those that contain a genome of interest and another partial or full genome of interest.

FIG. 5B depicts another mass spectrum 410 resulting from execution of step 108 of the process 100 in which the temperature of the simulated virus preparation 16 was elevated stepwise in temperature from 25° C. to 55° C. for a simulated incubation period of 20 minutes. In the illustrated example, the process 100 included step 110 in which, following expiration of the incubation period, the simulated virus preparation 16 was reduced stepwise in temperature from 55° C. back to 25° C. for execution of step 112 in which simulated measurements by the instrument 10 were carried out on the simulated, cooled virus preparation 16 with the instrument 10 operating at 25° C. Comparing the mass spectrum 410 to the mass spectrum 400 at step 114, it is clear from FIGS. 5A and 5B that the mass spectrum 410 exhibits an improvement in mass peak resolution of each capsid type over that of the mass spectrum 400. Moreover, as the resulting mass spectrum 410 does not appear to exhibit any peaks higher in mass than that attributable to the over-packaged capsids depicted in the mass spectrum 400, aggregation does not appear to be present in the mass spectrum 410. It should further be noted that whereas the mass peaks 402 and 406 of the empty and full capsids respectively appear to be more highly mass-resolved with stable or increased signal intensity, those of the partial and over-packaged capsids 404 and 408 respectively appear to be decreased in signal intensity as compared with the mass spectrum 400. In any case, step 116 advances to step 118 where, in this example, only the incubation period is changed by increasing it by 10 minutes. The temperature increase remains the same at 55° C.

The process steps 108-118 just described are repeated three additional times to subject the simulated virus preparation 16 to 55° C. at each three incrementally increased incubation periods of 30 minutes, 40 minutes and 60 minutes respectively. The resulting mass spectra 420 and 430 illustrated in FIGS. 5C and 5D respectively each exhibit an improvement in mass peak resolution of the empty 402 and full 406 capsids over that of the previously determined mass spectrum with no discernable aggregation. As FIGS. 5A-5D also demonstrate, the partial and over-packaged capsids begin, and continue, to disassemble at elevated temperatures with increasing incubation period duration as indicated by the decreasing intensities and ultimate disappearance in FIG. 5D of the mass peaks 404 and 408 of the partial and over-packaged capsids respectively under such conditions, indicating that such capsids are not stable under elevated temperature and respective incubation periods. It should be understood, however, that such instability of the partial and over-packaged capsids under the conditions depicted in FIGS. 5A-5D may be representative of the particular example virus preparation 16 used, but may not necessarily be representative of other types of virus preparations, and that in other types of virus preparations one or any combination of the capsid types may exhibit such instability while the remaining capsid types remain stable.

As further illustrated in FIG. 5E, while the resulting mass spectrum 440 likewise exhibits an improvement in mass peak resolution of the empty 402 and full 406 capsids over that of the previously determined mass spectrum 430, the mass spectrum 440 also exhibits high mass components 442 in the 6-7 MDa range, i.e., with mass greater than that of the original highest mass peak 408 attributable to over-packaged capsids, which is indicative of aggregation of at least some of the remaining empty and/or full virus capsids. Moreover, additional, lower-mass peaks 444, 446 and 488, e.g., between 0.2 and 2 MDa are also observed in the spectrum 440 depicted in FIG. 5E. The peak 444 is attributable to single-strand DNA resulting from the disassembly of some of the capsids, and the peak 446 is attributable to double-stranded DNA resulting from joining together of some of the single-stranded DNA. The peak 448 is attributable to proteins associated with the genomes of the disassembled partial and/or over-packaged capsids.

In the example execution of the process 100 depicted in FIGS. 5A-5E, the mass resolution of the peaks of interest improve with each incremental increase in incubation period at a common, elevated virus preparation temperature of 55° C., but the onset of aggregation appears to occur between incubation times of 40 minutes and 60 minutes. Accordingly, in this example execution of the process 100, the optimum combination of variables which minimize, or at least reduce, the heterogeneity of the virus preparation without aggregation is temperature=55° C. for a 40 minute incubation period. If heating profile is to be included in the optimum combination of variables, the heating profile in this particular example is a step-change, which in a practical application would correspond to control of the thermal energy source 24 to increase the temperature of the virus preparation from 25° C. to 55° C. as quickly as possible. If cooling profile is to be included in the optimum combination of variables, the cooling profile in this particular example is likewise a step-change, which in a practical application would correspond to quenching of the virus preparation, e.g., in an ice bath or other rapid-cooling environment. In any case, the optimum combination of variables in this example may be subsequently used in the process 300 depicted in FIG. 3 to heat-treat a similar virus preparation for the purpose of minimizing, or at least reducing, heterogeneity without aggregation of the remaining capsids.

While this disclosure has been illustrated and described in detail in the foregoing drawings and description, the same is to be considered as illustrative and not restrictive in character, it being understood that only illustrative embodiments thereof have been shown and described and that all changes and modifications that come within the spirit of this disclosure are desired to be protected.

1. A method for reducing heterogeneity of a virus preparation, the method comprising:
generating virus ions from the virus preparation,
repeatedly increasing at least one of a temperature and an incubation period at the increased temperature of at least one of the virus preparation and the generated virus ions,
measuring mass-to-charge ratios and charge magnitudes of at least some of the generated virus ions at each increase of the at least one of the temperature and the incubation period,
determining a mass spectrum at each increase of the at least one of the temperature and the incubation period based on values of the respective mass-to-charge ratios and charge magnitudes, and
determining, based on the mass spectrums, optimum ones of the temperature and the incubation period which together minimize, or at least reduce, a heterogeneity of the virus preparation without aggregation of virus capsids in the virus preparation.

2. The method of claim 1, further comprising:
varying a cooling profile corresponding to a manner in which the increased temperature is reduced following the respective incubation period, and
determining, based on the mass spectrums, an optimum cooling profile along with the optimum ones of the temperature and the incubation period which, in combination, minimize, or at least reduce, the heterogeneity of the virus preparation without aggregation of virus capsids in the virus preparation.

3. The method of claim 1, further comprising:
varying a heating profile corresponding to a manner in which the temperature of the at least one of the virus preparation and the generated virus ions is increased, and
determining, based on the mass spectrums, an optimum heating profile along with the optimum ones of the temperature and the incubation period which, in combination, minimize, or at least reduce, the heterogeneity of the virus preparation without aggregation of virus capsids in the virus preparation.

4. The method of claim 3, further comprising:
varying a cooling profile corresponding to a manner in which the increased temperature is reduced following the respective incubation period, and
determining, based on the mass spectrums, an optimum cooling profile along with the optimum heating profile and the optimum ones of the temperature and the incubation period which, in combination, minimize, or at least reduce, the heterogeneity of the virus preparation without aggregation of virus capsids in the virus preparation.

5. The method of claim 1, wherein measuring mass-to-charge ratios and charge magnitudes of at least some of the generated virus ions at each increase of the at least one of the temperature and the incubation period is carried out using a mass spectrometer or a charge detection mass spectrometer.

6. (canceled)

7. The method of claim 1, further comprising determining the heterogeneity of the virus population at each increase of the at least one of the temperature and the incubation period based on mass resolution of at least one mass peak of interest in the respective mass spectrum.

8. The method of claim 1, further comprising determining at each increase of the at least one of the temperature and the incubation period that aggregation has occurred if the respective mass spectrum includes discernable particles with masses greater than that of a highest mass capsid in the virus preparation,
wherein at least one of the optimum ones of the temperature and the incubation period is less than the respective temperature and incubation period of a respective mass spectrum in which aggregation has occurred.

9. The method of claim 1, further comprising treating other samples of the virus preparation to minimize, or at least reduce, heterogeneity thereof by heating each of the other samples of the virus preparation to the determined optimum temperature for the optimum incubation period.

10. The method of claim 1, wherein the virus preparation is a virus preparation solution,
and wherein generating the virus ions comprises generating the virus ions from the virus preparation solution using an electrospray ionization source.

11. The method of claim 1, wherein repeatedly increasing the at least one of the temperature and the incubation period comprises controlling a first thermal energy device coupled to the virus preparation to heat the virus preparation.

12. The method of claim 1, wherein repeatedly increasing the at least one of the temperature and the incubation period comprises controlling a second thermal energy device, positioned to transfer thermal energy to the generated ions, to heat the generated ions.

13. A method for reducing heterogeneity of a virus preparation, the method comprising:
sequentially increasing at least one of a temperature and an incubation period at the increased temperature of the virus preparation,
generating virus ions from the virus preparation at each increase of the at least one of the temperature and the incubation period,
measuring mass-to-charge ratios and charge magnitudes of at least some of the generated virus ions at each increase of the at least one of the temperature and the incubation period,
determining a mass spectrum at each increase of the at least one of the temperature and the incubation period based on values of the respective mass-to-charge ratios and charge magnitudes, and
determining, based on the mass spectrums, optimum ones of the temperature and the incubation period which together minimize, or at least reduce, a heterogeneity of the virus preparation without aggregation of virus capsids in the virus preparation.

14. The method of claim 13, further comprising:
varying a cooling profile corresponding to a manner in which the increased temperature is reduced following the respective incubation period, and
determining, based on the mass spectrums, an optimum cooling profile along with the optimum ones of the temperature and the incubation period which, in combination, minimize, or at least reduce, the heterogeneity of the virus preparation without aggregation of virus capsids in the virus preparation.

15. The method of claim 13, further comprising:
varying a heating profile corresponding to a manner in which the temperature of the virus preparation is increased, and
determining, based on the mass spectrums, an optimum heating profile along with the optimum ones of the temperature and the incubation period which, in combination, minimize, or at least reduce, the heterogeneity of the virus preparation without aggregation of virus capsids in the virus preparation.

16. The method of claim 15, further comprising:
varying a cooling profile corresponding to a manner in which the increased temperature is reduced following the respective incubation period, and
determining, based on the mass spectrums, an optimum cooling profile along with the optimum heating profile and the optimum ones of the temperature and the incubation period which, in combination, minimize, or at least reduce, the heterogeneity of the virus preparation without aggregation of virus capsids in the virus preparation.

17. The method of claim 13, wherein measuring mass-to-charge ratios and charge magnitudes of at least some of the generated virus ions at each increase of the at least one of the temperature and the incubation period is carried out using a mass spectrometer or a charge detection mass spectrometer.

18. (canceled)

19. The method of claim 13, further comprising determining the heterogeneity of the virus population at each increase of the at least one of the temperature and the incubation period based on mass resolution of at least one mass peak of interest in the respective mass spectrum.

20. The method of claim 13, further comprising determining at each increase of the at least one of the temperature and the incubation period that aggregation has occurred if the respective mass spectrum includes discernable particles with masses greater than that of a highest mass capsid in the virus preparation,
wherein at least one of the optimum ones of the temperature and the incubation period is less than the respective temperature and incubation period of a respective mass spectrum in which aggregation has occurred.

21. The method of claim 13, further comprising treating other samples of the virus preparation to minimize, or at least reduce, heterogeneity thereof by heating each of the other samples of the virus preparation to the determined optimum temperature for the optimum incubation period.

22. (canceled)

23. The method of claim 13, wherein sequentially increasing the at least one of the temperature and the incubation period comprises controlling a first thermal energy device coupled to the virus preparation to heat the virus preparation.
